(19) United States
(12) Patent Application Publication
(10) Pub. No.: US 2009/0104169 A1
(43) Pub. Date: Apr. 23, 2009

(54) PET FOOD COMPOSITION FOR SKIN PHOTOPROTECTION

(75) Inventors: Lionel Breton, Versailles (FR); Isabelle Bureau-Franz, Le Mont-Sur-Lausanne (CH)

Correspondence Address: BELL, BOYD & LLOYD LLP, P.O. Box 1135, CHICAGO, IL, 60690, US

(73) Assignee: Nestec S. A., Vevey (CH)

(21) Appl. No.: 12/338,677

(22) Filed: Dec. 18, 2008

Related U.S. Application Data
(63) Continuation of application No. 10/505,285, filed on Oct. 27, 2004, now abandoned.

(30) Foreign Application Priority Data
Feb. 21, 2002 (EP) 02075702.7

(51) Int. Cl. A61K 35/74

(52) U.S. Cl. 424/93.45

(57) ABSTRACT

An ingestable composition for the photoprotection of the skin of pets which comprises a photoprotecting effective amount of i) at least one probiotic lactic acid bacterium or a culture supernatant thereof, and ii) at least one yeast and/or a carotenoid or derivative, included into an ingestable carrier.

### CROSS REFERENCE TO RELATED APPLICATIONS

This application is a continuation of the U.S. application Ser. No. 10/505,285, filed Oct. 27, 2004, which is a continuation of the U.S. national stage designation of International application PCT/EP03/01687 filed Feb. 18, 2003, the content of which is expressly incorporated herein by reference thereto.

### FIELD OF THE INVENTION

The present invention relates to pet food composition, for the photoprotection of the skin, whether before, during and/or after exposure to UV radiation, and to the use of same for preventing and/or attenuating the damage caused by such UV irradiation. It also relates to a method to improve the photoprotection of the skin.

### BACKGROUND

The continuous decrease of the atmosphere's ozone layer with the concurrent increase of ultraviolet radiation reaching the planet's surface has attracted a great deal of interest in its potential consequence on human health.

Indeed, it is known that light radiation of wavelengths of from 320 nm to 400 nm (UV-A) promotes tanning of the human epidermis; such radiation, however, is likely to cause damage to the skin, especially in the case of sensitive skin or skin which is continuously exposed to solar radiation. UV-A rays cause, in particular, a loss in the elasticity of the skin and the appearance of wrinkles, promoting a premature aging thereof. It is also known to this art that light rays having wavelengths of from 280 to 320 nm (UV-B) cause erythema and skin burning which can impair the natural development of a tan.

Although exposure to ultraviolet radiation is needed to produce vitamin D, growing evidence suggests that extensive exposure to sun-light, in particular to ultraviolet radiation, causes a variety of problems in the skin, including induction of certain skin cancers and induction of accelerated skin ageing.

In addition to these established health concerns, research has also provided evidence suggesting that exposure to ultraviolet radiation may negatively affect a variety of immune responses in living beings both locally, within the UV-irradiated skin, and also systemically, i.e. at sites distant from the irradiated skin.

It is thus necessary, in order to maintain suitable skin quality after exposure to UV radiation, to prepare or treat the skin before the exposure, to protect it during the exposure and even to alleviate the detrimental effects of ultraviolet radiation on the skin, prevent the development of erythema, edema and/or flaking or scaling (hyperkeratosis) of the skin.

There is thus a need in the art for an orally administrable composition which is capable to improve and/or reinforce the photoprotective function of the skin of pets.

### SUMMARY

Accordingly, in a first aspect the present invention aims to provide an ingestable composition for the photoprotection of the skin which comprises a photoprotecting effective amount of i) at least one probiotic lactic acid bacteria or a culture supernatant thereof, and ii) at least one yeast, included into an orally acceptable carrier and/or one carotenoid or derivative, included into an ingestable carrier.

The present invention further relates to the use of a photoprotecting effective amount of i) at least one probiotic lactic acid bacteria or a culture supernatant thereof and ii) at least one yeast and/or one carotenoid or derivative, for preparing an ingestable carrier for protecting the skin of pets against radiations such as ultraviolet and all related skin disorders, such as erythema, inflammation, sun burn, barrier function, photoageing, alteration of the immune system, for example.

In a last aspect, the invention relates to a method for improving the photoprotective function of the skin of pets, which comprises the step of orally administering to the pet a composition comprising a photoprotecting effective amount of i) at least one probiotic lactic acid bacteria or a culture supernatant thereof, and ii) at least one yeast, in an ingestible carrier.

The invention also provides a method of reducing the effects of ageing in a pet comprising the step of feeding a pet a pet food composition as described above.

The combination according to the present invention has a particular beneficial effect on skin protection and colouration of the skin, that helps to reduce the effects of ultraviolet-related stress on skin.

Additional features and advantages of the present invention are described in, and will be apparent from, the following Detailed Description.

### DETAILED DESCRIPTION

Within the following description, “NCC” designates Nestle Culture Collection (Nestle Research Center, Vers-chez-les-Blanc, Lausanne, Switzerland). The term “photoprotection” is used to describe attempt to block or reduce the adverse clinical, histological and immunological effects of solar radiation exposure on the skin.

According to the present invention, the subject compositions comprise, as the active agents therefor, combinatory immixture of at least one probiotic lactic acid bacteria or bifidobacteria or a culture supernatant thereof, and at least one yeast and/or one carotenoid.

Indeed, it has now surprisingly and unexpectedly been determined that admixture of these very specific constituents elicits an enhanced effect or response in respect of the photoprotection of the skin.

Probiotics are non-pathogenic and non-toxigenic organisms, that survive passage through the stomach and small intestine. Upon continuous ingestion by the host they eventually may colonize the gut to a substantial extent thus competing with other potentially pathogenic bacteria for nutrients and/or attachment sites on the gastro-intestinal wall and reducing their numbers and reducing or preventing infections. Until now a number of different probiotic micro-organisms have been found, which all are reported to exert their effect in the gut via the production of toxins, metabolic by-products, short chain fatty acids and the like.

It has now been shown that probiotics do also exert an effect in an individual's body at a location distant from the region in which they colonize it. And particularly, it has been surprisingly found that a composition having a synergistic photoprotective effect on the skin may be obtained by combining into an ingestable carrier, a probiotic micro-organism and a yeast.

In a preferred embodiment, the probiotic to be included into the carrier is selected from the group consisting of lactic acid bacteria, in particular Lactobacilli and/or Bifidobacteria and are more preferably Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium infantis, Bifidobacterium adolescentis or Bifidobacterium pseudocatenulatum, or a mixture thereof.

According to a most preferred embodiment the strains Lactobacillus johnsonii NCC 533, Lactobacillus paracasei NCC 2461, Bifidobacterium adolescentis NCC 251 and Bifidobacterium longum NCC 490 were deposited by way of an example, under the Budapest Treaty with the Institut Pasteur (28 rue du Docteur Roux, F-75024 Paris cédex 15) on 30.06.92, 12.01.99, 15.04.99 and 15.03.99, respectively and under the deposit number CNCM I-1225, CNCM I-2116, CNCM I-2168 and CNCM I-2170, respectively.

The probiotic microorganism according to the present invention may be included in a live form, semi-active or in deactivated form, e.g. as a lyophilized powder. Also culture supernatants of the microorganisms may be included in the products, optionally in concentrated form. It may also be included in an encapsulated form. When using a supernatant of a probiotic's culture the supernatant may be used as such or may be subjected to one or more purification steps prior to inclusion into the product, so as to concentrate or isolate the active ingredient (s)/metabolite (s). Method and techniques for purifying compounds and detecting the activity thereof in the fractions obtained are well known to the skilled person.

The probiotic lactic acid bacteria may be present in the carrier in an amount of at least 10⁵ cfu/g of ingestable carrier, preferably from about 10⁵ to 10¹⁵ cfu/g of ingestable carrier, and more preferably from 10⁷ to 10¹² cfu/g of ingestable carrier.

It may be incorporated in dispersion form in a suitable vehicle such as water, organic solvents and fatty substances including oils, whether alone or in admixture.

The compositions according to the invention also comprise a yeast. In a preferred embodiment, the yeast is any food-grade yeast selected from the group consisting of Ascomycotina or Deuteromycotina. In a preferred embodiment, the yeast may be selected from the group consisting of Debaryomyces, Kluyveromyces, Saccharomyces, Yarrowia, Zygosaccharomyces, Candida and Rhodutorula, and more preferably Saccharomyces caerevisae (baker's yeast).

Such yeast may be used in the form of dried or lyophilized extracts. It may be present in the carrier in an amount of at least 10⁵ cfu/g of ingestable carrier, preferably from about 10⁵ to 10¹⁵ cfu/g of ingestable carrier, and more preferably from 10⁷ to 10¹² cfu/g of ingestable carrier, said amount depending on the nature and activity of the particular yeast.

The carotenoid may be a carotenoid with or without provitamin A activity. It may be β-carotene, γ-carotene, α-carotene, lycopene, zeaxanthine and luteine, or a mixture thereof. The carotenoid may be from synthetic or natural origin or contained in a natural extract. When the carotenoid is from natural origin, it is preferably obtained from plant material, in which the plant is grown in-vivo or in-vitro. Method for extracting the carotenoids are well known in the art. The carotenoid may be present in the carrier in an amount of from 10⁻¹²% to 20% by weight and preferably from 0.00001 mg to 50 mg/day and more preferably from 0.001 mg to 30 mg/day.

A mixture of a plurality of lactic acid bacteria, yeast and/or carotenoids may also be used.

The carrier may be any pet food or pharmaceutical product, or a nutritional supplement, wherein the probiotic microorganism, the yeast and/or carotenoid may be included. Methods for preparing the carrier are common knowledge.

The said composition may be administered to the pet as a supplement to its normal diet or as a component of a nutritionally complete pet food. It may also be a pharmaceutical composition.

The nutritionally complete pet food composition according to the invention may be in powdered, dried form, a treat or a wet, chilled or shelf stable pet food product. These pet foods may be produced by ways known in the art. Apart from the plant or plant extract, these pet foods may include any one or more of a starch source, a protein source and a lipid source.

Suitable starch sources are, for example, grains and legumes such as corn, rice, wheat, barley, oats, soy, and mixtures of these. Suitable protein sources may be selected from any suitable animal or vegetable protein source; for example meat and meal, poultry meal, fish meal, soy protein concentrates, milk proteins, gluten, and the like. For elderly animals, it is preferred for the protein source to contain a high quality protein. Suitable lipid sources include meats, animal fats and vegetable fats.

The choice of the starch, protein and lipid sources will be largely determined by the nutritional needs of the animal, palatability considerations, and the type of product applied. For elderly pets, the pet food preferably contains proportionally less fat than pet foods for younger pets. Furthermore, the starch sources may include one or more of rice, barley, wheat and corn.

The pet food may optionally also contain a prebiotic or another active agent, for example a long chain fatty acid. The amount of prebiotic in the pet food is preferably less than 10% by weight. For example, the prebiotic may comprise about 0.1% to about 5% by weight of the pet food. For pet foods which use chicory as the source of the prebiotic, the chicory may be included to comprise about 0.5% to about 10% by weight of the feed mixture; more preferably about 1% to about 5% by weight.

Suitable long chain fatty acids include linoleic acid, alpha-linolenic acid, gamma linolenic acid, eicosapentanoic acid, and docosahexanoic acid. Fish oils are a suitable source of eicosapentanoic acids and docosahexanoic acid. Borage oil, blackcurrant seed oil and evening primrose oil are suitable sources of gamma linoleic acid. Safflower oils, sunflower oils, corn oils and soybean oils are suitable sources of linoleic acid.

If necessary, the pet food is supplemented with minerals and vitamins so that they are nutritionally complete. Further, various other ingredients, for example, sugar, salt, spices, seasonings, flavouring agents, and the like may also be incorporated into the pet food as desired.

For dried pet food a suitable process is extrusion cooking, although baking and other suitable processes may be used. When extrusion cooked, the dried pet food is usually provided in the form of a kibble. If a prebiotic is used, the prebiotic may be admixed with the other ingredients of the dried pet food prior to processing. A suitable process is described in European patent application No 0850569. If a probiotic microorganism is used, the organism is preferably coated onto or filled into the dried pet food. A suitable process is described in European patent application No 0862863.

For wet food, the processes described in U.S. Pat. Nos. 4,781,939 and 5,132,137 may be used to produce simulated meat products. Other procedures for producing chunk type products may also be used; for example cooking in a steam oven. Alternatively, loaf type products may be produced by emulsifying a suitable meat material to produce a meat emulsion, adding a suitable gelling agent, and heating the meat emulsion prior to filling into cans or other containers.

In another aspect, the invention relates to a method for improving the photoprotective function of the skin of pets, which comprises the step of orally administering to the pet a composition comprising a photoprotecting effective amount of i) at least one probiotic lactic acid bacteria or a culture supernatant thereof, and ii) at least one yeast and/or a carotenoid or its derivative, in an ingestible carrier.

The amount of the composition to be consumed by the individual will depend on the desirable effect. However, an amount of the composition to provide a daily amount of about 10⁵ to 10¹² organisms, which organism may be alive or dead, and 0.00001 to 50 mg of carotenoids would usually be adequate.

The composition is administered to an individual before or during the exposure to ultraviolet radiations, in particular exposure to sun. When the exposure period is foreseeable, it is desirable to start the consumption of the composition from 10 to 20 days before, and to prolong consumption during exposure.

In order to further illustrate the present invention and the advantages thereof, the following specific examples are given, it being understood that same are intended only as illustrative and in nowise limitative. In said examples to follow, as in the above description, all parts and percentages are given by weight, unless otherwise indicated.

### EXAMPLES

### Example 1

### Dry Dog Food

A feed mixture is made up of about 58% by weight of corn, about 5.5% by weight of corn gluten, about 22% by weight of chicken meal, 2.5% dried chicory, salts, vitamins and minerals making up the remainder.

The fed mixture is fed into a preconditioner and moistened. The moistened feed is then fed into an extruder-cooker and gelatinised. The gelatinised matrix leaving the extruder is forced through a die and extruded. The extrudate is cut into pieces suitable for feeding to dogs, dried at about 110° C. for about 20 minutes, and cooled to form pellets. At this point, a lyophilized powder of one strain of the following strains: CNCM I-1225, CNCM I-2116, CNCM I-2168 or CNCM I-2170 and lyophilized powder of S. cerevisae (so that the corresponding amount of each is about 1.0E+05-1.0E+12 cfu/day) can be added and mixed to the product. Furthermore, the same amount of bacteria can be sprayed on the cooled pellets and dried at 50-60° C. for some minutes.

This dry dog food helps to protect the skin's natural defenses of pets against the sun's harmful UV rays.

### Example 2

### Canned Pet Food and Supplement

A mixture is prepared from 73% of poultry carcass, pig lungs and beef liver (ground), 16% of wheat flour, 2% of dyes, vitamins, and inorganic salts. This mixture is emulsified at 12° C. and extruded in the form of a pudding which is then cooked at a temperature of 90° C. It is cooled to 30° C. and cut in chunks. 45% of these chunks are mixed with 55% of a sauce prepared from 98% of water, 1% of dye, and 1% of guar gum. Tinplate cans are filled and sterilized at 125° C. for 40 min. As a probiotic supplement to be mixed with the pet-food before serving, additional packaging (e.g. sachet) with one of the following strains: CNCM I-1225, CNCM I-2116, CNCM I-2168 or CNCM I-2170, and β-carotene and lyophilized S. cerevisae. The corresponding amount for the pet is about 10⁵-10¹² cfu/day microorganisms and 0.1 to 50 mg of carotenoids, which can be supplied as a supplement with (e.g. on top of) the can.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1-29. (canceled)

30. A nutritionally complete pet food comprising an ingestible carrier having 1) at least one component selected from the group consisting of a probiotic lactic acid bacterium, a culture supernatant of a probiotic lactic acid bacterium, and combinations thereof, present in an amount of at least 10⁵ cfu/g, 2) at least one yeast present in an amount of at least 10⁵ cfu/g, and 3) at least one synthetic or natural carotenoid with or without provitamin A activity present in an amount of from about 10⁻¹²% to 20% by weight.
